(19) United States
(12) Patent Application Publication
(10) Pub. No.: US 2006/0228390 A1
(43) Pub. Date: Oct. 12, 2006

(54) Intravitreal implant

(76) Inventor: David F. Williams, Minnetonka, MN (US)

Correspondence Address: SCHWEGMAN, LUNDBERG, WOESSNER & KLUTH, P.A., P.O. BOX 2938, MINNEAPOLIS, MN, 55402, US

(21) Appl. No.: 11/124,799

(22) Filed: May 9, 2005

Related U.S. Application Data
(60) Provisional application No. 60/669,064, filed on Apr. 7, 2005.

(51) Int. Cl. A61F 2/06

(52) U.S. Cl. 424/422; 623/1.11

(57) ABSTRACT

A device and method for drug delivery is shown. In particular, a drug delivery device for implanting in an eye is shown. Advantages of drug delivery devices shown include an increased surface area allowing an increased drug dosage from a delivery mechanism such as a drug-containing coating. Another advantage of drug delivery devices shown includes a sharpened end portion allowing an incision to be made using the drug delivery device itself.

### RELATED APPLICATION

The present invention claims the benefit of priority under 35 U.S.C. 119(e) to United States Provisional Application 60/669,064, filed on Apr. 7, 2005, which is incorporated herein by reference in its entirety.

### TECHNICAL FIELD

This invention relates to devices and methods to treat medical conditions of the eye. Specifically, this invention relates to devices and methods for drug delivery within the eye.

### BACKGROUND

For any of a variety of medical treatments, it is desirable to introduce a drug to the interior of the eye. Drug delivery to treat conditions inside the eye, such as on the retina of the eye, is more effective when the drug is actually delivered inside the eye. Other treatments, such as an injection into the bloodstream, or drug delivery on the surface of the eye, are not as effective.

One method of delivering drugs includes coating an implant device with a coating, where the coating includes a dosage of the drug. The drug is transmitted from the coating into the vitreous fluid of the eye, and from the vitreous fluid, the drug is transmitted directly to the retina. One limitation of this method is that the available dosage of the drug per unit time is dependent on the surface area that is coated. A higher surface area in contact with the vitreous fluid leads to a higher available drug dosage per unit time.

What is needed is an improved design of a drug delivery implant device for intravitreal delivery. What is also needed is a device and method with a higher capability for drug dosage delivery per unit time.

### BRIEF DESCRIPTION OF THE DRAWINGS

FIG. 1A shows a side view of an intravitreal implant according to an embodiment of the invention.

FIG. 1B shows an end view of an intravitreal implant according to an embodiment of the invention.

FIG. 1C shows a top view of an intravitreal implant according to an embodiment of the invention.

FIG. 1D shows an intravitreal implant in place in an eye according to an embodiment of the invention.

FIG. 1E shows an intravitreal implant in place in an eye according to another embodiment of the invention.

FIG. 2 shows an end view of an intravitreal implant according to an embodiment of the invention.

FIG. 3 shows a side view of an intravitreal implant according to an embodiment of the invention.

FIG. 4 shows a side view of a portion of an intravitreal implant according to an embodiment of the invention.

FIG. 5 shows a side view of a portion of an intravitreal implant according to an embodiment of the invention.

FIG. 6 shows a side view of a portion of an intravitreal implant according to an embodiment of the invention.

FIG. 7 shows a side view of an intravitreal implant according to an embodiment of the invention.

### DETAILED DESCRIPTION

In the following detailed description, reference is made to the accompanying drawings which form a part hereof, and in which is shown, by way of illustration, specific embodiments in which the invention may be practiced. In the drawings, like numerals describe substantially similar components throughout the several views. These embodiments are described in sufficient detail to enable those skilled in the art to practice the invention. Other embodiments may be utilized and structural, mechanical or logical changes, etc. may be made without departing from the scope of the present invention.

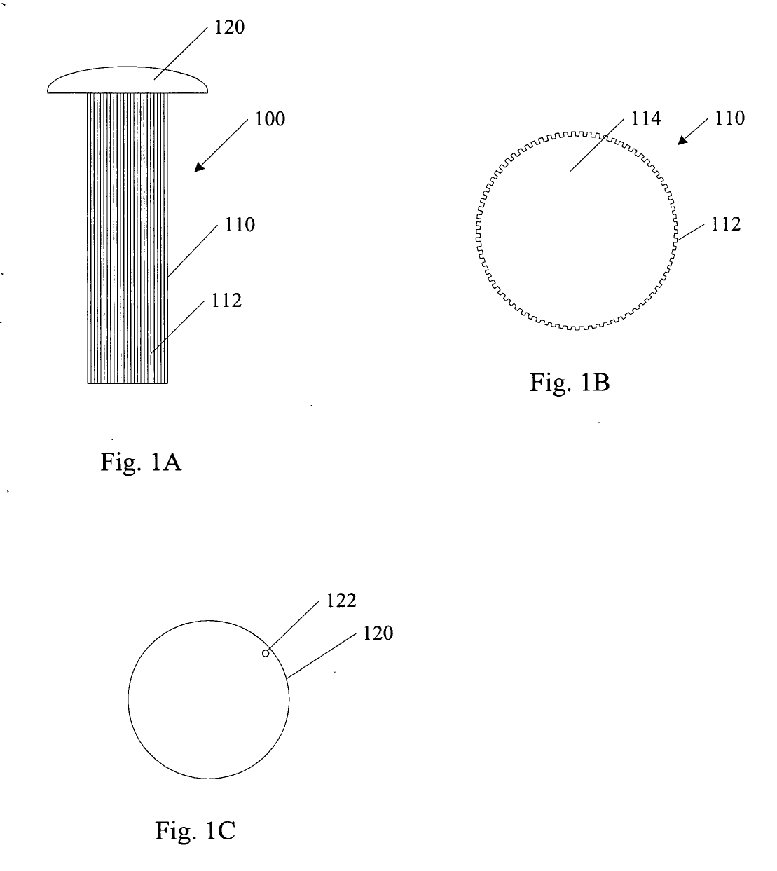

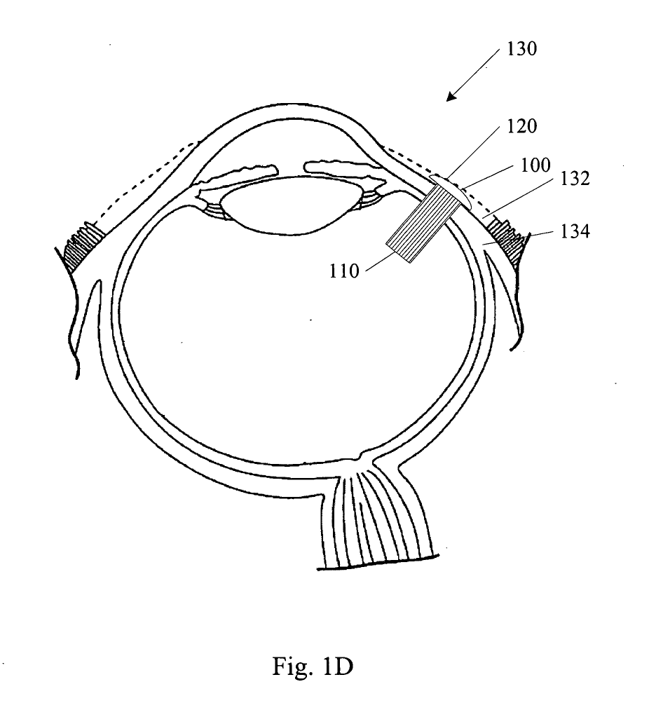

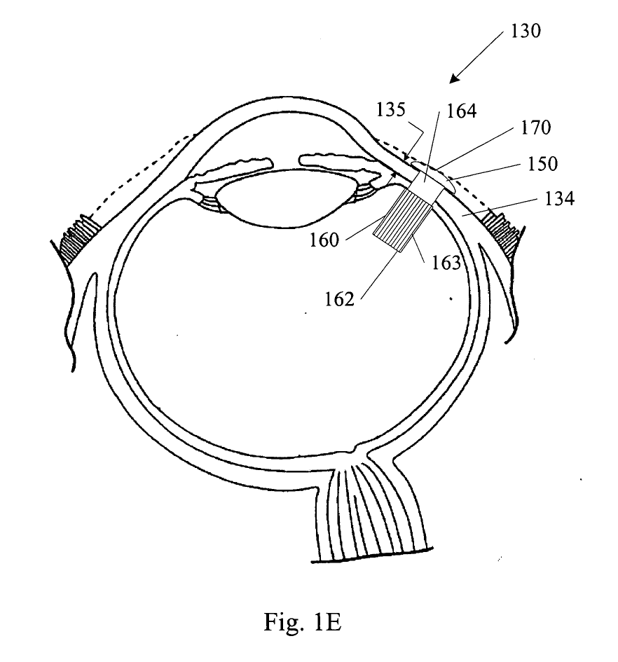

FIG. 1 shows a drug delivery device 100. The drug delivery device 100 includes a body portion 110 and a holding portion 120. Along a surface of the body portion, a number of undulating features 112 are shown. In one embodiment, the body portion 110 includes a cylindrical portion with a solid center 114 as shown in the end view in FIG. 1B. In one embodiment, the number of undulating features 112 include fluted features as can be seen in the end view of FIG. 1B. The fluted features shown in FIG. 1B include substantially square features, although the invention is not so limited. In other embodiments, alternative fluting geometries such as triangular, hemispherical, arcuate, etc. are possible.

In one embodiment, a coating that is adapted to accept a drug is included on an exposed surface of the body portion 110. In use, the drug delivery device 100 is implanted in a region, such as in an eye. Once in place, a drug that is contained on or within the coating is released into the region to be treated. Embodiments including undulating features 112 such as fluting increase an exposed surface area of the body portion 110. By increasing or decreasing the exposed surface area of the body region, a dosage of drug can be controlled. In embodiments using undulating features 112 such as fluting, a maximum available dosage is increased. In one example, design features such as flute geometry, number of flutes, size of flutes, etc. are utilized to control dosage of drug delivery. Although fluting is used as an example, other types of undulating features 112 can also be used to increase and control an available surface area of the body region for drug delivery.

In one coating embodiment, the body portion 110 is fabricated from a structural material or metal such as stainless steel, titanium, platinum, nickel-titanium alloy, etc. An example coating for drug delivery includes one or more polymers. Other drug delivery coatings available to one of ordinary skill in the art are also within the scope of the invention. In one embodiment, instead of a coating, the body portion 110 and undulating features 112 are fabricated entirely from a material capable of delivering a drug.

In one embodiment an attachment feature is included on the holding portion 120. In one embodiment, the attachment method includes suturing the holding portion to adjacent tissue. In one embodiment, one or more suture features 122 are included as shown in FIG. 1C.

FIG. 1D shows an example of one embodiment of a drug delivery device 100 in place within an eye 130. In one embodiment, the holding portion 120 is positioned adjacent to the sclera 134, and under the conjunctiva 132. The body portion 110 is shown with an exposed surface area within the vitreous chamber.

FIG. 1E shows an example of another embodiment of a drug delivery device 150 in place within an eye 130. Similar to the embodiment shown in FIG. 1D, a holding portion 170 is positioned adjacent to the sclera 134, and under the conjunctiva. A body portion 160 is shown in FIG. 1E with an exposed surface area within the vitreous chamber. The body portion 160 in FIG. 1E includes a distal portion 162 that includes a number of undulating features 163 similar to other embodiments described. The body portion 160 further includes a proximal portion 164 that is substantially smooth. In one embodiment, the proximal portion 164 includes a length that is approximately equal to or longer than a thickness 135 of the sclera 134. In one embodiment, while a number of undulating features 163 provide increased surface area, the substantially smooth proximal portion 164 provides a better matched interface between the sclera tissue and the body portion 160. One advantage of embodiments that include a substantially smooth proximal portion 164 includes reduced leakage from an incision after placement of the drug delivery device 150.

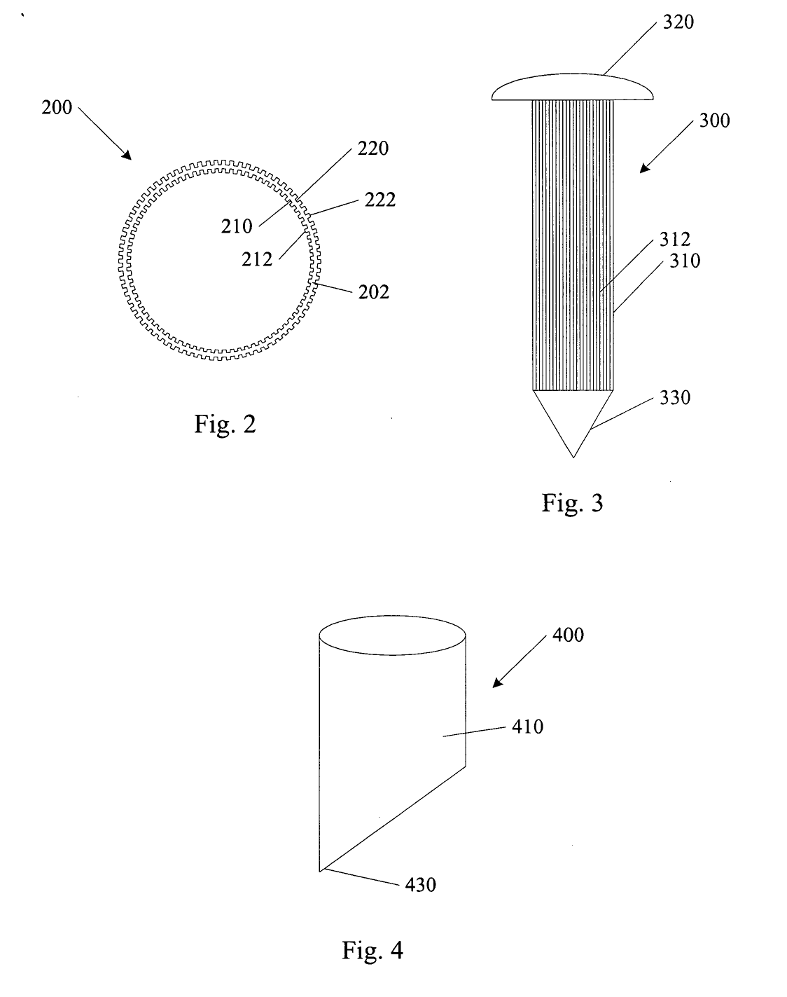

FIG. 2 shows an end view of a drug delivery device 200 according to one embodiment. The drug delivery device 200 includes a tubular configuration with an interior wall 210 and an exterior wall 220. In one embodiment, one or more walls include undulating features. In the embodiment shown in FIG. 2, the interior wall 210 includes fluting 212 and the exterior wall 220 also includes fluting 222. A solid portion 202 is shown between the interior wall 210 and the exterior wall 220. A tubular configuration such as shown in FIG. 2 can be used to further increase an available drug delivery surface area. In one embodiment, an end or ends of the tubular configuration are closed and the interior chamber is used to hold a dosage of drug that is transmitted through the solid portion 202 through diffusion or other openings.

FIG. 3 shows a side view of a drug delivery device 300 according to one embodiment. A body portion 310 and a holding portion 320 are shown similar to embodiments described above. Likewise, a number of undulating features 312 such as flutes are shown. In one embodiment, a sharpened portion 330 is included at an end of the body portion 310. The sharpened portion 330 is used to create an opening or incision in tissue such as the eye as described above.

FIG. 4 shows a portion of a drug delivery device 400 including a body portion 410. In one embodiment, a sharpened portion 430 is created by cutting the body portion 410 at an angle. Other embodiments include a sharpened portion that is separately attached to the body portion by welding, bonding, or other suitable fastening methods.

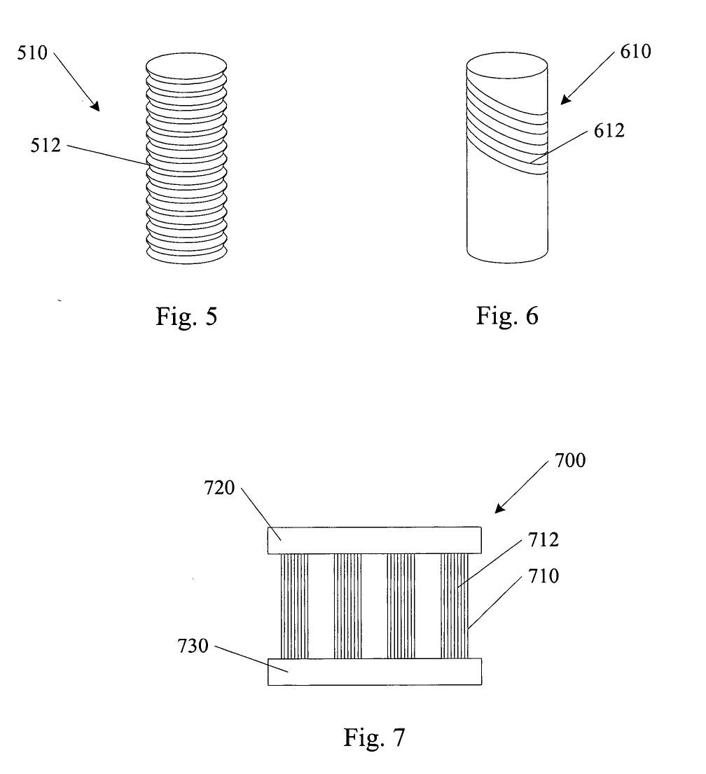

FIG. 5 shows an embodiment of a body portion 510 with a number of undulating features 512. As shown in FIG. 5, in one embodiment fluting or other undulating features 512 are oriented perpendicular to an axis of the body portion 510.

FIG. 6 shows an embodiment of a body portion 610 with a number of undulating features 612. In one embodiment, the undulating features 612 such as flutes are oriented in a tapered thread type configuration. A tapered thread configuration has the ability to increase a surface area of the body portion in a controlled manner through flute geometry, number of flutes, size of flutes, etc. The tapered thread configuration also has the ability to aid in insertion and retention of the body portion within tissue. In one embodiment (not shown) the body portion is formed in a complex shape such as a corkscrew shape and a number of undulating features as described above are included on an exposed surface of the corkscrew shaped body portion. Similar to the embodiment described above, the corskcrew shape aids in device retention, while the undulating features such as flutes can be added and varied to adjust/increase drug delivery surface area. Other body portion shapes are also within the scope of the invention. For example, in one embodiment the body portion includes an S-shape, or a C-shape, etc. In one embodiment, other body portion shapes include undulating features as described in embodiments above.

FIG. 7 shows a drug delivery device 700 according to an embodiment of the invention. Multiple body portions 710, including undulating features 712 formed using designs as described above, are included in the drug delivery device 700. In the embodiment shown in FIG. 7, a cage like structure is formed using a top portion 720 and a bottom portion 730. The number of body portions can be selected in addition to flute design options to further increase or select an available surface area for drug delivery.

### CONCLUSION

Thus has been shown a device for drug delivery using an implant and method of device use. Advantages of devices described above include an increased surface area allowing an increased drug dosage from a delivery mechanism such as a drug-containing coating. Another advantage of selected embodiments includes a sharpened end portion allowing an incision to be made using the drug delivery device itself. Variables of undulating features such as a flute geometry, number of flutes, size of flutes, etc. are adjusted to provide a desired drug delivery dosage per unit time.

While a number of advantages of embodiments described herein are listed above, the list is not exhaustive. Other advantages of embodiments described above will be apparent to one of ordinary skill in the art, having read the present disclosure. Although specific embodiments have been illustrated and described herein, it will be appreciated by those of ordinary skill in the art that any arrangement which is calculated to achieve the same purpose may be substituted for 30 the specific embodiments shown. This application is intended to cover any adaptations or variations of the present invention. It is to be understood that the above description is intended to be illustrative, and not restrictive. Combinations of the above embodiments, and other embodiments will be apparent to those of skill in the art upon reviewing the above description. The scope of the invention includes any other applications in which the above structures and methods are used. The scope of the invention should be determined with reference to the appended claims, along with the full scope of equivalents to which such claims are entitled.

I claim:

1. A drug delivery device, comprising:
a body portion for placement adjacent to an area to be treated;
a holding portion to locate the delivery device adjacent to a tissue surface; and
a plurality of undulating features on a surface of the body portion.

2. The drug delivery device of claim 1, wherein the plurality of undulating features includes a number of fluted features.

3. The drug delivery device of claim 2, wherein the body portion is elongated along an insertion axis.

4. The drug delivery device of claim 3, wherein the body portion includes a cylindrical body portion wherein the insertion axis is a central axis of the cylinder.

5. The drug delivery device of claim 1, wherein the body portion includes a substantially smooth portion adjacent to the holding portion.

6. The drug delivery device of claim 4, wherein the number of fluted features includes fluted features substantially parallel with the central axis.

7. The drug delivery device of claim 4, wherein the number of fluted features includes fluted features substantially perpendicular to the central axis.

8. The drug delivery device of claim 4, wherein the number of fluted features includes tapering threaded features along the cylinder.

9. The drug delivery device of claim 3, wherein the body portion includes a tubular body portion having an inside surface and an outside surface.

10. The drug delivery device of claim 9, wherein the plurality of undulating features is included on both the inside surface and the outside surface.

11. The drug delivery device of claim 1, further including a sharpened distal feature for penetrating tissue.

12. The drug delivery device of claim 1, wherein the holding portion includes a securing feature to fasten the holding portion to the adjacent tissue surface.

13. The drug delivery device of claim 1, wherein multiple body portions are included in an assembly, each body portion including a plurality of undulating features on a surface of the body portion.

14. A drug delivery device, comprising:
a body portion for placement adjacent to an area to be treated;
a holding portion to locate the delivery device adjacent to a tissue surface;
a plurality of flutes on a surface of the body portion; and
a sharpened distal feature for penetrating tissue.

15. The drug delivery device of claim 14, wherein the body portion includes a substantially smooth portion adjacent to the holding portion.

16. A method of intravitreal drug delivery, comprising:
selecting an implant device having a number of undulating features, wherein characteristics of the undulating features determine an implant device surface area;
inserting the implant device in a portion of an eye to be treated; and
releasing a drug, wherein the implant device surface area corresponds to desired drug delivery variables.

17. The method of claim 16, wherein inserting the implant device in a portion of an eye includes making an incision with a sharpened end portion of the implant device.
